(19) United States
(12) Patent Application Publication
(10) Pub. No.: US 2016/0067166 A1
(43) Pub. Date: Mar. 10, 2016

(54) COSMETIC COMPOSITION COMPRISING A NATURAL ORGANIC OIL AND POLYMETHYLMETHACRYLATE PARTICLES

(71) Applicant: DSM IP ASSETS B.V., The Heerlen (NL)

(72) Inventors: Achim BROCK, Basel (CH); Christian GSTOETTMAYR, Basel (CH); Aline HUEBER, Basel (CH); Szilvia MESAROS, Basel (CH); Juergen H. VOLLHARDT, Basel (CH)

(21) Appl. No.: 14/787,569

(22) PCT Filed: May 14, 2014
(86) PCT No.: PCT/EP2014/059821

§ 371 Date: Oct. 28, 2015

(30) Foreign Application Priority Data
May 16, 2013 (EP) 13168058.9

(51) Int. Cl. A61K 8/81; A61K 8/06; A61Q 19/04; A61Q 19/00; A61Q 19/08; A61Q 5/12; A61K 8/97; A61K 8/37

(52) U.S. Cl. CPC A61K 8/8147; A61K 8/97; A61K 8/064; A61K 8/375; A61Q 19/00; A61Q 19/08; A61Q 19/005; A61Q 19/004; A61Q 5/12; A61Q 19/04; A61K 2800/49; A61K 2800/74; A61K 2800/654; A61K 2800/596; A61K 2800/412

(57) ABSTRACT

The present invention relates to a topical composition comprising a natural organic oil and a porous cross-linked polymethylmethacrylate bead having a particle size Dᵥ0 of greater 0.3 μm, a Dᵥ100 of less than 35 μm and a Dᵥ50 selected in the range of 6 to 15 μm and an oil absorption capacity selected in the range of 1.2-2.5 cc/g. Furthermore the invention relates to the use of such polymethylmethacrylate beads to ameliorate the short and long-term sensory properties of a topical composition comprising a natural organic oil.

The present invention relates to a topical composition comprising a natural organic oil and a porous cross-linked polymethylmethacrylate bead having a particle size Dᵥ0 of greater 0.3 μm, a Dᵥ100 of less than 35 μm, a Dᵥ50 selected in the range of 6 to 15 pm and an oil absorption capacity selected in the range of 1.2-2.5 cc/g. Furthermore the invention relates to the use of such polymethylmethacrylate beads to ameliorate the short and long-term sensory properties of a topical composition comprising a natural organic oil.

Skin care compositions comprising natural organic oils for nourishing and caring of the skin are well known to a person skilled in the art. However such compositions often exhibit unpleasant sensory properties such as an oily, greasy feeling, imparting unwanted gloss to the skin, and rendering the composition sticky. Furthermore, they are often not easily absorbed or leave an off feeling of the absorbed product on the skin.

Even though texturing agents on the basis of polyamide, nylon, silica, polymethylmethacrylate or starch are known which claim to improve the sensory properties of topical compositions in general, these texturing agents commonly are employed in high amounts such as in amounts of 8% or more. Thus, there is still an ongoing need to further improve the complex sensory properties of topical compositions comprising natural organic oils in order to fulfill the demanding expectations on the part of the end consumers which is economical and thus does not need such high amounts of a texturing agent.

Surprisingly it has been found that the addition of relatively low amounts of a specific texturing agent to a topical composition comprising a natural organic oil significantly improves the short but also the long-term sensory properties.

Thus, the present invention relates to a topical composition comprising a natural organic oil, characterized in that the composition further comprises a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition.

Another subject matter of the invention is directed to a method for improving the sensory properties of a topical composition comprising a natural organic oil, said method comprising the step of adding to the topical composition a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition and appreciating the effect.

In a further embodiment the invention relates to the use of a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition to improve the sensory properties of a topical composition comprising a natural organic oil.

Preferably, the improved sensory properties are an increased waxiness and a reduced oiliness and greasiness resulting in an overall more velvet skin feel.

In all embodiments of the present invention preferably the oil absorption capacity is selected in the range of 1.5-2.0 cc/g.

The oil absorption capacity refers to the weight of a specific oil absorbed by a material, determined by a specific method as outlined in the following. It includes the oil absorption capacity of the dry particles existing between the inherent voids within and on the surface of the particles. The oil absorption capacity as referred to in the present invention is determined at 23° C. by weighting 2 g of the respective beads into a 20 ml beaker glass. Then, liquid paraffin (Paraffinum Perliquidum PH.EUR. CAS 8042-47-5) is added. After addition of 4 to 5 drops of paraffin to the powder, mixing is performed using a spatula, and addition of paraffin is continued until conglomerates of oil and powder have formed. From this point, the paraffin is added one drop at a time and the mixture is then triturated with the spatula. The addition of oil is stopped when the loose and dry powder completely disappears and a highly viscous white to transparent homogeneous gel is obtained. The oil absorption capacity (cc/g) is then calculated by the volume of paraffin used (in cc) per g of the respective beads.

The term natural organic oils as used according to the present invention relates to natural oils and fats (including butters) derived from animal, vegetable, or mineral sources. Preferred natural organic oils according to the invention are modern cosmetic oils known to be safe for cosmetic purposes such as almond oil, apricot kernel oil, argan oil, avocado butter, avocado oil, cocoa butter (theobroma oil), camelina oil, canola oil, carrot seed oil, castor oil, citrus seed oil, coconut oil, corn oil, cottonseed oil, cucumber oil, egg oil, grapeseed oil, hemp seed oil, jojoba oil, lanolin oil, linseed oil, macadamia nut oil, meadowfoam seed oil, mineral oil, mink oil, olive oil, palm kernel oil, peach kernel oil, peanut oil, rapeseed oil, rose hip oil, safflower oil, sesame oil, shark liver oil, shea butter, soybean oil, sunflower seed oil, sweet almond oil, tallow (beef) oil, tallow (mutton) oil, turtle oil, vegetable oil, and wheat germ oil as well as mixtures thereof. Particularly preferred natural organic oils according to the present invention are plant derived oils and fats such as in particular almond oil, apricot kernel oil, argan oil, avocado butter, avocado oil, cocoa butter (theobroma oil), camelina oil, canola oil, carrot seed oil, castor oil, citrus seed oil, coconut oil, corn oil, cottonseed oil, cucumber oil, grapeseed oil, hemp seed oil, jojoba oil, lanolin oil, linseed oil, macadamia nut oil, meadowfoam seed oil, olive oil, palm kernel oil, peach kernel oil, peanut oil, rapeseed oil, safflower oil, sesame oil, soybean oil, sunflower seed oil, sweet almond oil, vegetable oil and wheat germ oil as well as mixtures thereof. The most preferred natural organic oils according to the present invention are argan oil and sweet almond oil (Prunus Amygdalus Dulcis Oil) as well as mixtures thereof.

The amount of the natural organic oil is preferably selected in the range of 0.1-15 wt.-%, such as more preferably in the range of 0.25 to 10 wt.-%, and most preferably in the range of 0.5 to 5 wt.-%.

In all embodiments of the present invention, the amount of the porous polymethylmethacrylate bead is preferably selected in the range of 0.5 to 4 wt.-%, such as in particular in the range of 1.5 to 3.5 wt.-% based on the total weight of the composition.

The particle size (in volume %) as given in the present invention is determined by a Coulter LS13320 or Malvern Mastersizer 2000 according to standard methods in the art.

The porous polymethylmethacrylate beads according to the present invention are preferably obtained by copolymerization of a monomer mixture consisting of methyl methacrylate and ethylene glycol dimethacrylate in the presence of a porogen according to known methods in the art and as e.g. outlined in KR 2006036614 which is enclosed herein by reference.

The term ‘consisting of’ as used according to the present invention means that the total amount of monomers ideally sums up to 100 wt.-%. It is however not excluded that small amount of impurities or additives may be present such as e.g. in amounts of less than 5 wt.-%, preferably less than 3 wt.-% which are e.g. introduced via the respective raw materials.

The porogen is preferably selected from the group consisting of toluene, n-hexanone, methylisobutyl ketone and isoamyl alcohol.

Initiators for polymerizing the monomers to provide the porous polymethylmethacrylate beads of the invention are those which are normally suitable for free-radical polymerization of acrylate monomers and which are oil-soluble and have low solubility in water such as e.g. organic peroxides, organic peroxyesters and organic azo initiators. The initiator is generally used in an amount of about 0.01 to 1 wt.-% based on the total monomer content.

Optionally, a water soluble inhibitor can be added to inhibit polymerization in the water phase in order to prevent the formation of too much polymer by emulsion and/or solution polymerization in the water phase, which can result in bead agglomeration or emulsion type polymerization. Suitable inhibitors include those selected from thiosulfates, thiocyanates, water soluble hydroquinones and nitrites. When used, the water soluble inhibitor can generally be added in an amount of from about 0.01 to about 1 parts by weight based on 100 parts total monomer content.

Furthermore, a water soluble or water dispersible polymeric stabilizer is needed to stabilize the suspension and in order to obtain stable beads. The stabilizer is preferably a water soluble or water dispersible polymer such as e.g. polyvinylpyrrolidone, polyvinylmethylether, polyethyleneimine, polyvinylalcohol, gelatin, starch, (m)ethylcellulose, carboxymethylcellulose, hydroxyethylcellulose, hydroxypropyl ellulose, poly(meth)acrylic acid and their sodium salts, and the like. The stabilizer is preferably used in an amount of about 0.001 to 10 wt.-%, more preferable in an amount of about 0.01 to 1 wt-% based on the total monomer content.

The monomers, free-radical initiator, and any optional materials can be mixed together in the prescribed ratio to form a premix. The stabilizer can be combined with water and then with the premix to form an oil in water suspension. The resulting suspension typically comprises from about 10 to about 50 weight percent monomer premix and from about 90 to about 50 weight percent water phase. Bead-type suspension polymerization in accordance with the present invention is typically a thermally initiated polymerization and is preferably carried out with agitation for about 2 to about 16 hours at a temperature between about 40° C. and 90° C.

After isolation of the beads according to standard methods such as filtration or centrifugation the beads are preferably washed e.g. with water and/ or ethanol and subjected to an extended drying, preferably at about 40-100° C. and more preferably at about 80-100° C. in order to further reduce the residual monomer content to an amount of below 250 ppm, such as in particular below 100 ppm, and even more particular below 50 ppm. The drying can be performed by commonly known means to a person skilled in the art such as e.g. using a fluidized bed dryer or a conventional oven. The drying time can be easily adjusted by a person skilled in the art and is usually carried out over a period of 3 to 40 h such as about 8 to 20 h and in particular about 8 to 10 h.

In all embodiments of the present invention the porous polymethylmethacrylate beads are preferably prepared by suspension polymerisation of a monomer mixture consisting of 10-90 wt.-% methyl methacrylate and 10-90 wt.-% ethylene glycol dimethacrylate, with the proviso that the sum of monomers sums up to 100 wt.-%, in the presence of a porogen selected from toluene, n-hexanone, methylisobutyl ketone and isoamyl alcohol and a stabilizer selected from the group consisting of polyvinyl pyrrolidone, polyvinylmethylether, polyethyleneimine, poly(acrylicacid), polyvinylalcohol, vinyl acetate copolymer and ethyl cellulose.

Particularly suitable porous polymethylmethacrylate beads according to the present invention have a Dᵥ50 selected in the range of 9 to 12 μm and an oil absorption capacity selected in the range of 1.5-2.0 cc/g. Furthermore, it is preferred that the residual monomer content is less than 100 ppm, preferably below 50 ppm (determined by Gas Chromatography). It is furthermore advantageous if the beads exhibit as 10% aqueous dispersion in distilled water a pH in the range of 5.0-9.0. It is furthermore preferred if the porous polymethylmethacrylate beads have a water content of less than 1.5 wt.-% (determined by Karl Fischer titration).

Suitable porous polymethylmethacrylate beads according to the present invention having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g are e.g. commercially available as VALVANCE™ Touch 150 at DSM Nutritional Products Ltd Kaiseraugst.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibers. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations or functional preparations.

Examples of skin care preparations are, in particular, light protective preparations (sunscreens), anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), as well as skin lightening preparations as well as BB and CC Creams.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are skin care preparations, such as (body) milks, lotions, hydrodispersions, foundations, creams, creamgels, serums, toners or gels.

The topical compositions according to the present invention may be in the form of a suspension or dispersion in solvents or fatty substances, or alternatively in the form of an emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. oil-in-water-in oil (O/W/O) or water-in-oil-in-water (W/O/W) type), pickering emulsion, hydrogel, alcoholic gel, lipogel, one- or multiphase solution or vesicular dispersion or other usual forms, which can also be applied by pens, as masks or as sprays.

In one embodiment, the topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W, respectively Si/W emulsifier is preferably used in an amount of 0.5 to 10 wt.-%, in particular in the range of 0.5 to 6 wt.-% such as more in particular in the range of 0.5 to 5 wt.-% such as most in particular in the range of 1 to 4 wt.-%, based on the total weight of the composition.

Particular suitable O/W emulsifiers according to the present invention encompass phosphate esters emulsifier of formula (II)

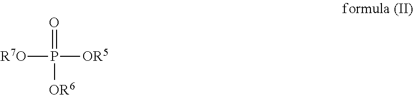

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular advantageous phosphate ester emulsifier according to the invention is potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate. Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

In particular embodiment, the invention relates to topical compositions in the form of O/W emulsions comprising an oily phase dispersed in an aqueous phase in the presence of an O/W emulsifier wherein the O/W emulsifier is selected from the group consisting of potassium cetyl phosphate, glyceryl stearate (and) PEG-100 Stearate, cetearyl olivate and sorbitan olivate as well as mixtures thereof.

In another particular embodiment, the invention relates to topical compositions in the form of W/O emulsions comprising an aqueous phase dispersed in an oily phase in the presence of a W/O emulsifier.

Suitable W/O emulsifiers encompass polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS [INCI Name Polyglyceryl-4 Diisostearate/Polyhydroxystearate/Sebacate (i.e. diester of a mixture of isostearic, polyhydroxystearic and sebacic acids with Polyglycerin-4)] or Dehymuls PGPH available at Cognis (INCI Polyglyceryl-2 Dipolyhydroxystearate).

Particularly preferred according to the present invention are W/O emulsions wherein the W/O emulsifier is Polyglyceryl-3 Diisostearate.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W or W/O emulsions according to the invention can be provided, for example, in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the topical compositions constitute cosmetic composition and are intended for topical application to the skin.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into such compositions. Such cosmetic ingredients commonly used in the skin care industry, which are suitable for use in the compositions of the present invention, are e.g. described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7.5. The pH can easily be adjusted as desired with suitable acids such as e.g. citric acid or bases such as sodium hydroxide (e.g. as aqueous solution), Triethanolamine (TEA Care), Tromethamine (Trizma Base) and Aminomethyl Propanol (AMP-Ultra PC 2000) according to standard methods in the art.

The amount of the topical composition to be applied to the skin is not critical and can easily be adjusted by a person skilled in the art. Preferably the amount is selected in the range of 0.1-3 mg/cm² skin, such as preferably in the range of 0.1 to 2 mg/cm² skin and most preferably in the range of 0.5 to 2 wt.-%/cm².

The following examples are provided to further illustrate the compositions and effects of the present invention. These examples are illustrative only and are not intended to limit the scope of the invention in any way.

### EXAMPLE

To 30 g of Nivea rich Body milk (Beiersdorf, Art. 80203, Ingredients: Aqua, Paraffinum Liquidum, C13-16 Isoparaffin, Glycerin, Isopropyl Palmitate, Cera Microcristallina, PEG-40 Sorbitan Perisostearate, Polyglyceryl-3 Diisostearate, Glyceryl Glucoside, Prunus Amygdalus Dulcis Oil, Maris Sal, Magnesium Sulfate, Sodium Citrate, Citric Acid, Potassium Sorbate, Linalool, Limonene, Benzyl Alcohol, Geraniol, Citronellol, Butylphenyl Methylpropional, Benzyl Salicylate, Cinnamyl Alcohol, Alpha-Isomethyl Ionone, Hydroxycitronellal, Hexyl Cinnamal, Parfum; W/O emulsion) in a 100 mL beaker 3 wt.-% of the respective texturing agent as outlined in table 1 was added under homogenization for 1 minute.

TABLE 1
Tested texturing agents:

| Sample | Texturing agent |
|---|---|
| 1 | Sepimat P (INCI: Methyl Methacrylate Crosspolymer) |
| | solid polymethylmethacrylate beads having a D50 of |
| | 5-15 μm from Seppic |
| 2 | Orgasol Caressse (INCI: Polyamide-5) from Arkema |
| 3 | NAVIANCE ® TAPIOCA P (INCI: Tapioca |
| | Starch) from Akzo Nobel |
| 4 | MSS-500 (INCI: Silica) from Kobo Products |
| 5 | DRY-FLO PC (INCI: Aluminum Starch Octenylsuccinate) |
| | from Akzo Nobel |
| 6 | Orgasol 2002 EXD Nat Cos (INCI: Nylon-12) from Arkema |
| 7 | VALVANCE ™ Touch 150 (INCI: Methyl Methacrylate |
| | Cross Polymer) porous polymethylmethacrylate beads according |
| | to the invention from DSM Nutritional Products Ltd Kaiseraugst. |

The samples were tested in a blind study with a trained sensorial panel consisting of 6 persons under the following conditions:

The evaluation takes part on the inner forearm; the panel leader applies 50 μL of the respective sample.

Evaluator spreads the product within a defined circle of 5 cm diameter using index or middle finger, circular motion, rate of 2 rotations/second. This is the so called rub-out phase. After the rub-out phase the immediate after-feel as well as the 20 minutes after-feel was assessed according to standardized parameters.

The intensities felt are quantified on a scale from 0 to 100 in comparison to training standards with known and defined sensory intensities.

The results are given as difference of the values determined for the tested sample and the plain benchmark, i.e. the Nivea rich Body milk without texturing agent (Δ=value sample−value plain benchmark).

A decreased absorbency during the rub-out phase is advantageous as this indicates a faster application. Furthermore it is preferred if the sample exhibits a reduced gloss, a reduced oiliness and an increased waxiness as this results in an overall more dry and velvet skin feel.

| | rub-out | | | immediate afterfeel | | | | 20 minutes afterfeel | | | |
|---|---|---|---|---|---|---|---|---|---|---|---|
| | Oil | Wax | Abs. | Gloss | Oily | Waxy | Greasy | Gloss | Oily | Waxy | Greasy |
| | Δ AV | | | Δ AV | Δ % | Δ % | Δ % | Δ % | Δ % | Δ % | Δ % |
| 1 | −2.1 | −1.0 | 12.1 | −4.8 | −9.5 | 0.0 | 0.5 | 0.4 | −0.7 | −4.4 | 4.6 |
| 2 | −5.3 | −0.6 | 2.2 | −8.2 | −10.0 | 0.3 | 0.6 | −2.5 | −0.7 | 5.8 | −5.5 |
| 3 | −5.2 | 1.7 | −3.6 | −9.9 | −10.2 | 5.2 | 0.2 | −2.2 | −0.7 | −4.2 | 2.2 |
| 4 | −0.2 | 0.0 | 2.6 | −2.6 | 0.0 | 2.8 | 3.3 | −0.8 | 0.1 | −4.2 | 1.9 |
| 5 | −2.0 | −1.3 | 4.1 | −9.4 | −9.7 | 0.2 | 3.8 | −0.3 | −0.7 | −2.0 | 7.1 |
| 6 | −5.3 | 0.9 | −2.9 | −11.5 | −12.5 | 2.5 | 0.2 | 6.1 | −0.6 | −1.5 | −0.4 |
| 7 | −9.8 | 7.0 | −12.7 | −19.4 | −17.4 | 12.8 | −0.8 | −3.9 | −0.7 | 15.0 | −7.8 |

Rub out phase (absolute values (AV))

Oil: evaluated after 15-20 rubs, defined as “amount of oil felt between fingertip and skin” (Light Mineral Oil=100)

Wax: evaluated after 15-20 rubs, defined as “amount of wax felt between fingertip and skin” (Beeswax-type pomade=90)

Absorbency (Abs.): defined as “The number of rubs at which product loses wet, moist feel and a resistance to continue is perceived”, upper limit is 120 rubs (Nivea Cream=40, Light Mineral Oil=120+)

Immediate and 20 min after-feel

Gloss: amount of light reflected off skin (untreated skin=10), absolute value (AV)

Oily: percentage of perceived oily character (liquid, fatty) of the residue

Waxy: percentage of perceived waxy character (dry, dull) of the residue

Greasy: percentage of perceived greasy character (solid, fatty) of the residue

Formulation Examples

1. Facial Care Cream

| Phase | Ingredients | INCI Name | Wt.-% |
|---|---|---|---|
| A | Olivem 1000 | CETYL ALCOHOL | 2.5 |
| | Lanette 16 | CAPRYLIC/CAPRIC | 4.0 |
| | | TRIGLYCERIDE | |
| | Tegosoft CT | | 6.0 |
| | Fitoderm ® | SQUALANE | 3.0 |
| | Macadamia nut oil | MACADAMIA TERNIFOLIA | 2.0 |
| | | SEED OIL | |
| B | Preservative | | q.s. |
| | Glycerin 99.5% | GLYCERIN | 3.0 |
| | Keltrol CG-T | XANTHAN GUM | 0.30 |
| | WATER DEM. | AQUA | ad 100 |
| C | Naviance Tapioca | TAPIOCA STARCH | 2.0 |
| | ALPAFLOR ® | LEONTOPODIUM ALPINUM | 2.5 |
| | EDELWEISS EP | EXTRACT, GLYCERIN, | |
| | | AQUA, CITRIC ACID, | |
| | | SODIUM BENZOATE, | |
| | | POTASSIUM SORBATE | |
| | ARGAN OIL | ARGANIA SPINOSA | 2.5 |
| | | KERNEL OIL | |
| | VALVANCE ™ | Methyl Methacrylate | 2.50 |
| | Touch 150 | Cross Polymer | |

2. Anti-Aging Cream (O/W Emulsion)

| Ingredients | Wt.-% |
|---|---|
| Glyceryl Myristate | 4.00 |
| Cetyl Alcohol | 2.00 |
| Steareth-2 | 2.00 |
| Steareth-21 | 2.00 |
| Isopropyl Myristate | 5.00 |
| Caprylic/Capric Triglyceride | 8.00 |
| Argan Oil | 2.50 |
| BHT | 0.05 |
| Dimethicone | 2.00 |
| Phenoxyethanol & Methylparaben & Ethylparaben & | 0.80 |
| Butylparaben & Propylparaben & Isobutylparaben | |
| VALVANCE ™ Touch 150 (Methyl Methacrylate | 2.50 |
| Cross Polymer) | |
| Water | ad 100 |
| Xanthan Gum | 0.50 |
| Disodium EDETA | 0.10 |
| Propylene Glycol | 4.00 |

3. Anti-Acne Cream and Concealer

| Ingredients | INCI Name | Wt.-% |
|---|---|---|
| Emulgade | Cetearyl Glucoside and Cetearyl Alcohol | 2.00 |
| PL 68/50 | | |
| Lanette E | Sodium Cetearyl Sulfate | 0.25 |
| Tegin 4100 | Glyceryl Stearate | 1.00 |
| Pellets | | |
| Cetiol OE | Dicaprylyl Ether | 4.00 |
| Shea Butter | Butyrospermum Parkii | 2.00 |
| Cetiol PGL | Hexyldecanol (and) Hexyldecyl Laurate | 3.00 |
| Jojobaoil | Jojobaoil | 1.00 |
| DC 345 | Cyclopentasiloxane and Cyclohexasiloxane | 0.50 |
| Rapithix A 100 | Sodium Polyacrylate | 0.50 |
| Water | Aqua | ad 100 |
| Glycerin | Glycerin | 2.00 |
| Euxyl PE 9010 | Phenoxyethanol and Ethylhexylglycerin | 1.00 |
| SA Timiron | Titanium Dioxide, Mica, 2% Dimethicone, | 5.00 |
| Silk Green | Tin dioxide | |
| VALVANCE ™ | Methyl Methacrylate Crosspolymer | 3.00 |
| Touch 150 | | |
| NaOH 10% | Sodium Hydroxide | 2.30 |

4. Cream for Baby Care and After Sun Cream

| Ingredients | INCI Name | Wt.-% |
|---|---|---|
| Imwitor 372P | Glyceryl Stearate Citrate | 2.00 |
| Cutina GMS | Glyceryl Stearate | 3.00 |
| Sympatens-O/4200 | Sorbitan Laurate & Polyglyceryl-10 | 1.00 |
| Sweet Almond Oil | Prunus Amygdalus Dulcis | 2.50 |
| Tegosoft TN | C12-15 Alkyl Benzoate | 7.00 |
| Cetiol OE | Dicaprylyl Ether | 5.00 |
| PENTAVITIN | Aqua, Saccharide Isomerate, | 5.00 |
| | Citric Acid | |
| Tegosoft DC | Decyl Cocoate | 3.00 |
| BHT | BHT | 0.05 |
| Euxyl PE 9010 | Phenoxyethanol & Ethylhexylglycerin | 1.00 |
| Dow Corning 345 | Cyclopentasiloxane and | 2.00 |
| | Cyclohexasiloxane | |
| Keltrol RD | Xanthan Gum | 0.30 |
| Glycerin | Glycerin | 2.00 |
| Water | Aqua | ad 100 |
| VALVANCE ™ | Methyl Methacrylate Crosspolymer | 2.00 |
| Touch 150 | | |
| Rapithix A-60 | Sodium Polyacrylate and Hydrogenated | 0.30 |
| | Polydecene and Trideceth-6 | |

Acid or Base if necessary to pH 5-6

5. Oil in Water Foundation for Sensitive Facial Skin

| Ingredients | INCI | Wt.-% |
|---|---|---|
| Deionised Water | Aqua | ad 100 |
| Glycerin | Glycerin | 2.00 |
| Triethanolamine | Triethanolamine | 0.80 |
| Paratexin | Methylparaben EP | 0.20 |
| Keltrol | Xanthan Gum | 0.30 |
| VALVANCE ™ Touch 150 | Methyl Methacrylate | 3.00 |
| | Crosspolymer | |
| Titanium dioxide | C.I. 77891 | 4.57 |
| SunCROMA yellow iron oxide | C.I. 77492 | 0.30 |
| SunCROMA red iron oxide | C.I. 77491 | 0.13 |
| SunCROMA black iron oxide | C.I. 77499 | 0.20 |
| DC 556 | Phenyl Trimethicone | 3.60 |
| Jojobaöl | Jojoba oil | 2.00 |
| Stearic Acid | Stearic Acid | 1.4 |
| Cetyl Alcohol | Cetyl Alcohol | 3.0 |
| Paratexin P | Propylparaben EP | 0.1 |

6. Water in Oil Cream Extra Care Against Stressed Skin

| Ingredients | INCI | Wt.-% |
|---|---|---|
| Cremophor WO-7 | PEG-7 Hydrogenated Castor Oil | 2.50 |
| Elfacos ST-9 | PEG-45/Dodecyl Glycol Copolymer | 2.00 |
| Cirebelle 303 | Synthetic Wax | 5.00 |
| Cirebelle 109L | Synthetic Wax | 7.20 |
| Miglyol 818 | Caprylic/Capric/Linoleic | 5.00 |
| | Triglyceride | |
| Eutanol G | Octyldodecanol | 7.50 |
| Cetiol OE | Dicaprylyl Ether | 6.00 |
| Avocado oil | Avocado oil | 2.00 |
| Deionised Water | Aqua | ad 100 |
| Glycerin | Glycerin | 5.00 |
| Propylene Glycol | Propylene Glycol | 2.00 |
| Euxyl PE 9010 | Phenoxyethanol and | 0.80 |
| | Ethylhexylglycerin | |
| VALVANCE ™ | Methyl Methacrylate Crosspolymer | 3.00 |
| Touch 150 | | |

7. Soothing Body Lotion (O/W Lotion) e.g. After Sun Care

| Ingredients | INCI | Wt.-% |
|---|---|---|
| Deionised Water | Aqua | ad 100 |
| Menthol | Menthol | 0.10 |
| Keltrol CG SFT | Xanthan Gum | 1.25 |
| Ceralution ES | Ceteareth-25, Di Sodium Ethylene | 2.00 |
| | Dicocamide PEG-15 Disulfate | |
| Isofol 20 | Octyldodecanol | 5.00 |
| Sweet Almond Oil | Prunus Amygdalus Dulcis | 3.50 |
| Paratexin EC5 | Benzoic Acid Benzyl Alcohol, | 1.00 |
| | Dehydroacetic Acid, Sorbic Acid | |
| VALVANCE ™ | Methyl Methacrylate Crosspolymer | 2.00 |
| Touch 150 | | |

8. Leave-in Hair and Scalp Conditioner and After Sun Treatment

| Ingredients | INCI | Wt.-% |
|---|---|---|
| Deionised Water | Aqua | ad 100 |
| Ethanol DEB 96 | Alcohol denat. | 30.00 |
| PVP/VA Copolymer | PVP/VA Copolymer | 2.50 |
| Euxyl K-300 | Phenoxyethanol, Methylparaben, | q.s. |
| | Butylparaben, Ethylparaben, | |
| | Propylparaben, Isobutylparaben | |
| Almond oil | Almond oil | 0.50 |
| Fragrance ADAM | Parfum | 0.10 |
| Triethanolamine 99% | Triethanolamine | 0.01 |
| FD & C Yellow No 5 | CI 19140, Aqua | 0.10 |
| (0.5% Solution) | | |
| FD & C Blue No 1 | CI 42090, Aqua | 0.10 |
| (0.5% Solution) | | |
| VALVANCE ™ | Methyl Methacrylate Crosspolymer | 3.00 |
| Touch 150 | | |

9. Skin Lightening Cream (O/W)

| Ingredients | Wt.-% |
|---|---|
| Estol 3650 (Glyceryl Myristate) | 5.00 |
| Lanette 16 (Cetyl Alcohol) | 2.00 |
| Tegosoft M (Isopropyl Myristate) | 10.00 |
| Vitamin E Acetate (Tocopheryl Acetate) | 0.50 |
| Almond Oil | 2.00 |
| BHT | 0.05 |
| Phenonip (Phenoxyethanol & Methylparaben & Ethylparaben | q.s. |
| & Propylparaben & Butylparaben & Isopropylparaben) | |
| Tris (Tromethamine) | 0.90 |
| EDETA BD (Disodium EDTA) | 0.10 |
| Propylene Glycol | 5.00 |
| VALVANCE ™ Touch 150 (Methyl Methacrylate | 2.00 |
| Crosspolymer) | |
| ALPAFLOR ® Edelweiss | 1.50 |
| Sepigel 305 (Polyacrylamide & C13-14 Isoparaffin | 2.00 |
| & Laureth-7) | |
| Triethanolamine | q.s. |
| Water deionized | ad 100 |

10. Skin Lightening Cream (O/W)

| Ingredients | Wt.-% |
|---|---|
| Estol 3650 (Glyceryl Myristate) | 5.00 |
| Lanette 16 (Cetyl Alcohol) | 2.00 |
| Brij 72 (Steareth-2) | 2.00 |
| Brij 721 (Steareth-21) | 2.00 |
| Tegosoft M (Isopropyl Myristate) | 10.00 |
| Bisabolol | 0.20 |
| Vitamine E Acetate (Tocopheryl Acetate) | 1.00 |
| Almond Oil | 2.00 |
| BHT | 0.05 |
| Phenonip (Phenoxyethanol & Methylparaben & Ethylparaben | q.s. |
| & Propylparaben & Butylparaben & Isopropylparaben) | |
| EDETA BD (Disodium EDTA) | 0.10 |
| Propylene Glycol | 10.00 |
| Sodium Ascorbyl Phosphate | 1.00 |
| Arbutin | 1.00 |
| D-Panthenol | 0.50 |
| Niacinamide | 0.20 |
| Salicylic acid | 0.50 |
| VALVANCE ™ Touch 150 (Methyl Methacrylate | 1.50 |
| Crosspolymer) | |
| Edelweiss extract | 0.5 |
| Sepigel 305 (Polyacrylamide & C13-14 Isoparaffin & | 2.00 |
| Laureth-7) | |
| Triethanolamine | q.s. |
| Water deionized | ad 100 |

11. Skin Lightening Gel

| Ingredients | Wt.-% |
|---|---|
| Pemulen TR-1 (Acrylate/C10-30 Alkyl Acrylate | 0.80 |
| Crosspolymer) | |
| Jojoba oil | 3.00 |
| Biotin | 0.01 |
| EDETA BD (Disodium EDTA) | 0.10 |
| D- Panthenol | 0.20 |
| Hyasol BT (Sodium Hyaluronate) | 1.00 |
| Euxyl K 400 (Methyldibromo Glutaronitrile & | q.s. |
| Phenoxyethanol) | |
| NaOH (30%) | 1.00 |
| Propylene Glycol | 5.00 |
| VALVANCE ™ Touch 150 (Methyl Methacrylate | 1.50 |
| Crosspolymer) | |
| Niacinamide | 0.50 |
| Hydroquinone | 0.20 |
| Resveratrol | 0.1 |
| Edelweiss extract | 0.1 |
| Citric Acid (10%) | q.s. |
| Water deionized | ad 100 |

12. Light W/O Emulsion

| Phase | Ingredients | INCI Name | Wt.-% |
|---|---|---|---|
| A | Isolan GPS | Polyglyceryl-4 Diisostearate/ | 3.00 |
| | | Polyhydroxystearate/Sebacate | |
| | Tegosoft DEC | Diethylhexyl Carbonate | 7.00 |
| | DC 345 | Cyclopentasiloxane | 8.80 |
| | Shea Butter | Butyrospermum parkii | 3.00 |
| | Cutina HR | Hydrogenated Castor Oil | 0.10 |
| B | Glycerin | Glycerin | 2.00 |
| | Vitamin C | Aqua, Ascorbic Acid | 0.30 |
| | | Magnesium Sulfate Heptahydrate | 1.50 |
| | Water dem. | Aqua | ad 100 |
| C | VALVANCE ™ | Methyl Methacrylate Crosspolymer | 2.50 |
| | Touch 150 | | |
| | Parfum | Fragrance/Perfum | q.s. |
| | NaOH | Sodium hydroxide | q.s. |

14. Self-Tanning Lotion

| Phase | Ingredients | INCI Name | Wt.-% |
|---|---|---|---|
| A | Abil EM 90 | Cetyl PEG/PPG-10/1 | 2.00 |
| | | Dimethicone | |
| | Isopropylstearate | Isopropyl Stearate | 10.00 |
| | Mineral Oil | Mineral Oil | 10.00 |
| | Shea Butter | Butyrospermum parkii | 4.50 |
| | Microcristalline Wax | Microcristalline Wax | 0.40 |
| | Kakao Butter | Theobroma cacao (Cocoa) Seed | 2.00 |
| | | Butter | |
| | DL | Tocopherol Acetate | 0.50 |
| | Tocopherolacetat | | |
| B | Glycerin | Glycerin | 2.00 |
| | C*Pharmsorbidex | Sorbitol, Aqua | 3.00 |
| | NC 16205 | | |
| | Vitamin C | Aqua, Ascorbic Acid | 0.30 |
| | | Sodium Chloride | 0.50 |
| | Wasser | Aqua | Ad 100 |
| C | PEPHA ®-TIGHT | Aqua, Pullulan, Algae Extract, | 2.00 |
| | | Phenoxyethanol, Sodium | |
| | | Benzoate, Potassium Sorbate | |
| | DHA | Dihydroxyacetone | 5.00 |
| | Erythrulose | Erythrulose, Aqua | 2.00 |
| | VALVANCE ™ | Methyl Methacrylate | 2.00 |
| | Touch 150 | Crosspolymer | |
| | Euxyl PE 9010 | Phenoxyethanol, Ethylglycerin | q.s. |
| D | Parfum | Fragrance/Perfum | q.s. |
| | FD & C Blue 1 | FD & C Blue 1 | q.s. |
| | NaOH | Sodium Hydroxide | q.s. |

15. O/W Emulsion for Pregnant Woman

| Phase | Ingredients | INCI Name | Wt.-% |
|---|---|---|---|
| A | Olivem 1000 | Cetearyl Olivate (and) Sorbitan | 4.00 |
| | | Olivate | |
| | Kaffee Butter | Coffea arabica, Hydrogenated | 4.50 |
| | | Vegetable Oil | |
| | Kakao Butter | Theobroma cacao (Cocoa) Seed | 4.00 |
| | | Butter | |
| | Shea Butter | Butyrospermum parkii | 4.50 |
| | Jojobaöl | Simmondsia chinensis (Jojoba) | 5.00 |
| | | Seed Oil | |
| | Sweet almond | Prunus amygdalus dulcis | 5.00 |
| | Oil | (Sweet Almond) Oil | |
| | DL | Tocopherol Acetate | 0.50 |
| | Tocopherolacetat | | |
| B | Glycerin | Glycerin | 3.00 |
| | Sisterna L70-C | Aqua, Sucrose Laurate, Alcohol | 3.00 |
| | Euxyl 9010 | Phenoxyethanol, Ethylglycerin | 0.80 |
| | Keltrol | Xanthan gum | 0.40 |
| | C*Pharmsorbidex | Sorbitol, Aqua | 1.00 |
| | NC 16205 | | |
| | Wasser ad 100 | Aqua | 60.20 |
| C | ALPAFLOR ® | Glycerin, Aqua, Echinacea purpurea | 1.00 |
| | Echinacea AO | (Echinacea) Extract, Potassium | |
| | | Sorbate, Sodium Benzoate | |
| | VALVANCE ™ | Methyl Methacrylate Crosspolymer | 2.50 |
| | Touch 150 | | |
| D | Parfum | Fragrance/Perfum | 0.10 |
| | NaOH | Sodium hydroxide | q.s. |

16. Anti Stretchmarks Body Lotion for Teens

| Phase | Ingredients | INCI Name | Wt.-% |
|---|---|---|---|
| A | Pemulen TR-1 | Acrylates/C10-30 Alkyl | 0.60 |
| | | Acrylate Crosspolymer | |
| | Lexol EHS | Ethylhexyl Stearate | 8.00 |
| | Rose Hip Oil | Rosa Moschata | 10.00 |
| | DL Tocopherolacetat | Tocopheryl Acetate | 0.50 |
| | Euxyl 9010 | Phenoxyethanol, Ethylglycerin | 0.80 |
| B | Glycerin 86% | Glycerin | 2.00 |
| | Nicotinsäureamid | Niacinamide | 0.20 |
| | Kaffeine | Coffein | 0.20 |
| | Wasser | Aqua | ad 100 |
| C | NaOH, 10% | Sodium Hydroxide | 0.35 |
| D | VALVANCE ™ | Methyl Methacrylate | 1.50 |
| | Touch 150 | Crosspolymer | |
| E | Parfum | Fragrance | 0.20 |
| | Rot 1% | D&C Red 40 | 0.50 |
| | Blau 0.1% | FD&C Blue 1 | 0.20 |
| | Gelb 1% | FD&C Yellow 6 | 0.30 |

1. A topical composition comprising a natural organic oil, wherein the topical composition further comprises a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total amount of the composition.

2. The topical composition according to claim 1, wherein the amount of the porous polymethylmethacrylate bead is selected in the range of 0.5-4 wt.-% based on the total weight of the cosmetic composition.

3. The topical composition according to claim 1, wherein the particles size Dᵥ50 is selected in the range of 9 to 12 μm and the oil absorption capacity is selected in the range of 1.5-2.0 cc/g.

4. The topical composition according to claim 1, wherein the natural organic oil is a plant derived oil.

5. The topical composition according to claim 4, wherein the plant derived oil is selected from the group consisting of almond oil, apricot kernel oil, argan oil, avocado butter, avocado oil, cocoa butter (theobroma oil), camelina oil, canola oil, carrot seed oil, castor oil, citrus seed oil, coconut oil, corn oil, cottonseed oil, cucumber oil, grapeseed oil, hemp seed oil, jojoba oil, lanolin oil, linseed oil, macadamia nut oil, meadowfoam seed oil, olive oil, palm kernel oil, peach kernel oil, peanut oil, rapeseed oil, safflower oil, sesame oil, soybean oil, sunflower seed oil, sweet almond oil, vegetable oil and wheat germ oil as well as mixtures thereof.

6. The topical composition according claim 4, wherein the plant derived oil is selected from the group consisting argan oil and sweet almond oil as well as mixtures thereof.

7. The topical composition according to claim 1, wherein the amount of the natural organic oil is selected in the range of 0.1-15 wt.-% based on the total weight of the composition.

8. The topical composition according to claim 1, wherein the porous polymethylmethacrylate bead is obtained by copolymerization of a monomer mixture consisting of methyl methacrylate and ethylene glycol dimethacrylate in the presence of a porogen.

9. The topical composition according to claim 1, wherein the topical composition is in the form of a W/O emulsion comprising an aqueous phase dispersed in an oily phase in the presence of a W/O emulsifier.

10. The topical composition according to claim 9, wherein the W/O emulsifier is Polyglyceryl-3 Diisostearate.

11. The topical composition according to claim 9, wherein the composition is a body milk.

12. Use of a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition to improve the sensory properties of a topical composition comprising a natural organic oil.

13. A method for improving the sensory properties of a topical composition comprising a natural organic oil, said method comprising the step of adding to the topical composition a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition and appreciating the effect.

14. A use according to claim 12 or a method according to claim 13, wherein the improved sensory properties are an increased waxiness and a reduced oiliness and greasiness.
